(19) United States
(12) Patent Application Publication
(10) Pub. No.: US 2011/0046495 A1
(43) Pub. Date: Feb. 24, 2011

(54) DEVICE FOR MEASURING THE SIZE OF AN INTRACARDIAC OPENING

(76) Inventor: Peter Osypka, Rheinfelden-Herten (DE)

Correspondence Address: VOLPE AND KOENIG, P.C., UNITED PLAZA, 30 SOUTH 17TH STREET, PHILADELPHIA, PA, 19103, US

(21) Appl. No.: 12/854,406

(22) Filed: Aug. 11, 2010

(30) Foreign Application Priority Data
Aug. 21, 2009 (DE) 102009038500.2-35

(51) Int. Cl. A61B 5/02

(52) U.S. Cl. 600/508

(57) ABSTRACT

A device (1) for measuring the diameter of an intracardiac opening (2), having a hollow body (3) made from a metal-wire mesh is provided that has twisted individual bars that form the jacket of the discontinuous wall (5) of the hollow body (3) and form longitudinal stabilization between two elastically deformable metal-wire meshes (6) forming the end sides of the hollow body (3) and are deformable in a position of use by an edge (17) of the opening (2) in a contact region into the interior of the hollow body (3). Thus, with the device (1), the size of the opening (2) can be determined in that, in an X-ray or ultrasound image, the deformation of the individual bars (4) is comparable through the opening (2) with markings (19, 20) arranged on the hollow body (3) and/or on the device (1) adjacent to the hollow body (3).

### CROSS-REFERENCE TO RELATED APPLICATIONS

This application claims the benefit of German Patent Application No. 10 2009 038 500.2-35, filed Aug. 21, 2009, which is incorporated herein by reference as if fully set forth.

### BACKGROUND

The invention relates to a device for measuring the size of an intracardiac opening, with a hollow body having an elastic wall and able to be inserted into the opening and able to expand perpendicular to the direction of insertion, and with at least two transparent markings arranged at a predetermined distance to each other and whose distance can be compared with the molding of the hollow body by the edge of the opening to be measured, wherein the hollow body is permeable to liquid and is constructed as a metal cage.

A comparable device is known from US 2006/0173300 A1. The metal cage forming the hollow body should be pressed in here in a middle region by the edges of the opening to be measured, so that conclusions can be made on the size of the opening. Thus, the essentially uniformly constructed metal cage should be pressed in only in the region of the hole edges, but, incidentally, should remain stable, which is rarely achievable in such typical metal cages in practice. Instead it is to be expected that the middle region of this metal cage is deformed inward overall and that no clear and significant notch appears in the region of the edges of a hole to be measured.

A device of this type is known from DE 699 35 601 T2 in which a balloon mounted on the distal end of a catheter is brought into the opening to be measured and filled with a contrast means until a pressure threshold is reached or a left-right shunt can no longer be observed at the opening, wherein, for observing the left-right shunt, a Doppler echocardiograph is used. The size of the opening can be determined in that the balloon is again filled with the same quantity of supplied contrast means outside of the body of the patient and is positioned in different openings of a matrix. Furthermore, transparent markings can be formed on the shaft of the catheter.

It has been proven that the deformation of the balloon due to the edges of the examined opening in the heart is relatively difficult to recognize in an X-ray image, so that monitoring with a Doppler echocardiograph is required.

From DE 10 2007 018 763 U1, a balloon catheter for determining the size of an opening is known in which, with the help of an activation element, the expansion movement of the balloon can be converted into an adjustment movement running in the extension direction of the shaft of the catheter for a display element arranged outside of the body of the patient and surrounding the shaft in a ring shape. The expansion of the balloon takes place by inserting a liquid or by inflation.

### SUMMARY

Therefore, there is the objective of creating a device for measuring the size of an intracardiac opening in which contrast means are not required and in which the use of a balloon can be avoided and simple handling can be achieved, wherein the deformation of the hollow body in the X-ray or ultrasound image can be recognized easily and can be determined reliably through comparison with the markings, without the hollow body itself becoming deformed at undesired positions and the size determination of the opening becoming imprecise.

According to the invention, for meeting this objective a device of the type named above is provided in which the hollow body is discontinuous in its middle region between its ends and the discontinuity is spanned by individual bars that are each formed by at least two wires twisted with each other.

The twisted wires or wire sections provide stabilization directed, in particular, longitudinally, for the liquid-permeable hollow body that is adequately soft and flexible in the region of the opening to be measured, so that the dimensions of the edge of the opening can be easily recognized in the X-ray or ultrasound image with reference to the deformation of these twisted wires and wire sections. Simultaneously, the metallic ends can largely stabilize the hollow body.

Therefore, because the hollow body has a fluid-permeable construction, the patient is adversely affected as little as possible by the hollow body arranged in the opening in the position of use and not closing the opening and the balloon-less hollow body can be folded together again easily and compactly after use for removal, without which here a balloon would be an interference.

The handling and determination of the size of the opening is promoted when, in the starting position, the individual bars run parallel to each other and to a longitudinal center axis of the hollow body. Thus, the individual bars can be arranged on an imaginary lateral surface of a cylindrical region of the hollow body, so that an easily recognizable depression in the position of use in the region of an opening to be measured is produced.

Thus, the size of the opening can be determined through the comparison of the size of the molding of the metal cage by the edges of the opening with the distance of the markings in an X-ray or ultrasound image. In this way, the hollow body can be widened or expanded in the opening in a controlled way until the opening is filled up.

However, it has been proven that the hollow body can also be expanded in an uncontrolled way, because the deformation of the hollow body can be recognized easily through the opening in the X-ray or ultrasound image and allows a determination of the size of the opening. This simplifies the handling of the device even more.

It is further advantageous that structurally complicated guide elements for converting the widening movement of the hollow body into a display movement running in the extension direction of a shaft outside of the body of a patient are not required, because the size of the opening is easily recognizable with reference to the deformation of the hollow body in the X-ray or ultrasound image and can be determined reliably through comparison with the markings.

The expansion or widening of the hollow body in the radial direction can be achieved easily when the hollow body is made from an elastic material. It could also be provided that the hollow body is made from a memory metal. Here it is advantageous that the hollow body changes automatically into the expanded position due to the body heat of the patient in the position of use and thus unfolds itself. As the memory metal, any metal with shape memory that can be activated thermally, for example, nitinol, could be used.

Here it is preferable when the hollow body has, in the measurement region, several, in particular, at least two individual bars that describe a cylindrical lateral surface, and are sufficiently stable for stabilizing the hollow body and are soft and flexible in the contact region with the edge of the opening.

Here it is especially favorable when the individual bars run through the opening in the position of use and contact the edge of the opening. Here it is advantageous that the size of the opening can be evaluated with reference to the deformations from the straight shape of the individual bars.

According to one construction of the invention of standalone importance, it can be provided that the hollow body has wire sections that are twisted with each other and are in touching contact with the edge of the opening in the position of use. The twisted wire sections provide stabilization that is directed, in particular, longitudinally for the hollow body and is sufficiently soft and flexible in the region of the opening, so that the dimensions of the edge of the opening can be recognized easily in the X-ray or ultrasound image with reference to the deformation of the wire sections.

In order to achieve that the hollow body adapts automatically to the opening, it can be provided that the hollow body has, on each of its ends arranged on both sides of the opening in the position of use, a metal-wire mesh that can be deformed in the radial direction elastically against a restoring force from a maximum expanded position.

The restoring force can be applied by a spring force of the metal wires of the metal-wire mesh and/or it could be provided that the ends of the hollow body are connected by a connection element that is elastically deformable against a tensile stress and running advantageously centrally through the hollow body. Here, the metal-wire meshes are directed so that shortening of the connection element causes straightening of the metal-wire meshes and thus an increase in the extent of the hollow body.

Here it can be provided that the metal-wire mesh applies a spring force through which, in the relaxed position, the hollow body assumes its maximum extent in the region of the opening. This spring force can also be triggered by the body heat of the patient, with this heat causing a transition of the metal-wire mesh made, for example, from memory metal into an expanded form.

In order to be able to bring the hollow body through a narrow feed, for example, a blood vessel, into the position of use, an insertion catheter with a holder for the hollow body can be provided in the radially folded-together or compressed form. Advantageously, the holder is constructed with a sleeve-like shape.

It can be provided that the hollow body is mounted on a push-and-pull element and can be moved with the push-and-pull element into the holder and from the holder.

Simple handling of the device is produced when the holder is constructed on a shaft of the insertion catheter, in particular, on the distal end, holding the hollow body in the position of use.

According to one construction of the invention, it can be provided that the markings are arranged on a shaft holding the hollow body during its feed and during its use adjacent to the hollow body. Alternatively or additionally, the markings could be arranged adjacent to the hollow body on a push-and-pull element connected to the hollow body. Here it is advantageous that the markings are arranged in the vicinity of the opening on a part that does not change shape or changes shape slightly during use and thus are easily visible on the X-ray image or ultrasound image of the opening with the inserted hollow body for size comparison.

It can also be provided that the markings are arranged on the hollow body. In an especially favorable way, the arrangement on an individual bar has been proven. Here, a change in the distance of the markings from each other due to the deformation of the individual bar is insignificant, if the opening should be allocated only in one size class and therefore low demands are placed on the measurement accuracy.

The markings could be constructed, for example, as section with increased thickness.

For the free unfolding of the hollow body it can be provided that the hollow body is mounted so that it can move on a front end on the push-and-pull element. Advantageously, the other front end is arranged on the push-and-pull element rigidly, that is, unmovable.

According to one construction of the invention, it can be provided that the hollow body is connected detachably to the shaft and/or to the push-and-pull element. Here it is advantageous that the hollow body can be easily exchanged. Thus, different hollow bodies could also be used for measurements in different size regions on a device. Here it is especially favorable if the connection is constructed as a screw connection.

### BRIEF DESCRIPTION OF THE DRAWINGS

The invention will now be described in detail with reference to a preferred embodiment, but is not limited to this embodiment. Additional embodiments are given through the combination of features of the claims with each other and/or with individual or multiple features of the embodiments.

Shown in schematized diagrams are:

FIG. 1 is a view of a device according to the invention in an expanded or widened position,

FIG. 2 is a view of the device according to the invention according to FIG. 1 in a compressed position, and

FIG. 3 is a view of the device according to the invention according to

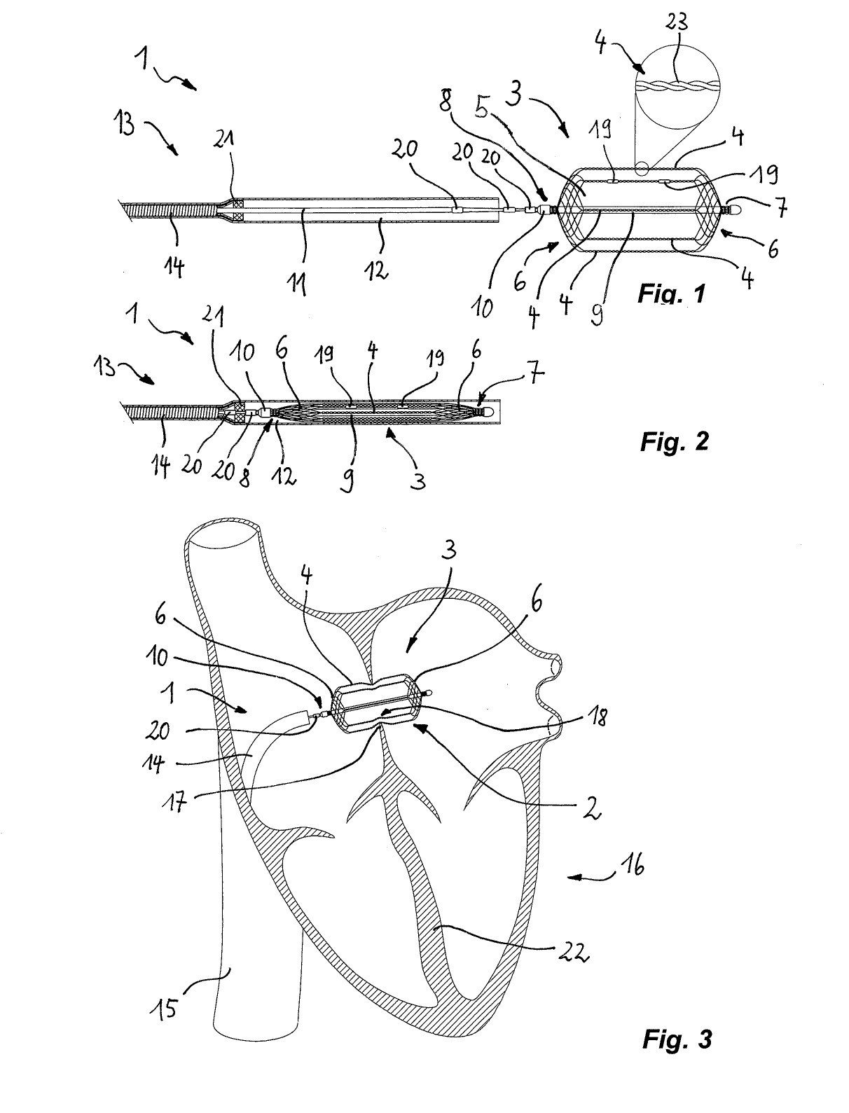

FIG. 1 in its position of use.

### DETAILED DESCRIPTION OF THE PREFERRED EMBODIMENTS

FIG. 1 shows partially in a section diagram a device designated as a whole with 1 that is constructed and designed for measuring the size of an intracardiac opening 2 visible in FIG. 3 and described in detail there.

The device 1 has a hollow body 3 that is constructed as a metal cage. The metal cage thus describes the discontinuous outer face of the hollow body 3.

The hollow body 3 is produced from an elastic material.

The hollow body 3 has multiple individual bars 4 that run approximately or precisely parallel and stabilize the hollow body 3 in the position shown in FIG. 1 and have a soft construction, however, perpendicular to their profile direction such that they can be easily deformed by pressure from the outside into the interior of the hollow body 3.

The individual bars 4 describe the lateral surface of a lateral surface that is approximately or precisely cylindrical and forms the wall 5 of the hollow body 3.

The cylindrical lateral surface of the wall 5 is bounded on both sides by a metal-wire mesh 6. These metal-wire meshes 6 are each formed from wires that are braided with each other and are twisted for forming the individual bars 4 in the middle region of the hollow body 3.

Through the twisting, the individual bars 4 that thus span the discontinuity of the hollow body 3 between the metal-wire meshes 6 obtain longitudinal strength through which the hollow body 3 is stabilized in its shape.

The individual bars 4 are each formed from two, three, or more wires 23 guided in parallel and twisted with each other. Therefore, because these wires 23 are produced integrally from the metal-wire meshes 6, in the individual bars 4, a spring force is produced that is directed outward and perpendicular to their extension direction.

Each metal-wire mesh 6 is connected at a front end 7, 8 of the hollow body 3 to a connection element.

The connection element 9 is elastically deformable in tension along its extension direction and can therefore produce a force on the metal-wire mesh 6 that acts against the individual bars 4, wherein unfolding of the metal-wire mesh 6 is achieved in the expanded or widened position of the hollow body 3 shown in FIG. 1.

The hollow body 3 is connected, via a screw connection 10, to a push-and-pull element 11 with which the hollow body 3 can be brought into a sleeve-shaped holder 12.

FIG. 2 shows the hollow body 3 in the position in the sleeve-shaped holder 12. In this way, through the dimensions of the inner diameter of the sleeve-shaped holder 12, it is achieved that the hollow body 3 is folded together and compressed in the radial direction, in order to fit into the sleeve-shaped holder 12.

Here, the metal-wire meshes 6 and optionally the connection element 9 are deformed elastically against a spring force, wherein the spring force causes the hollow body 3 to expand again into the position shown in FIG. 1 as soon as the hollow body 3 is pushed out from the sleeve-shaped holder 12 by the push-and-pull element 11.

In one embodiment, this spring force is built up or reinforced as soon as the hollow body 3 comes into thermal contact with a warm environment, for example, the body of a patient, in that the hollow body 3 is made from a metal with shape memory, in particular, nitinol. Here, the shape of the metal cage of the hollow body 3 stored in the shape memory is the expanded shape shown in FIG. 1.

In another embodiment, it can be provided that the connection element 9 has a rigid construction, wherein the front end 7 is fixed on the connection element 9, while the front end 8 on the connection element 9 is mounted so that it can move longitudinal to its extension direction.

The sleeve-shaped holder 12 is arranged on the distal end 13 of a shaft 14 of a vein catheter.

In order to reach the position of use of the device 1 shown in FIG. 3, the distal end 13 with the hollow body 3 arranged in the holder 12 is inserted through a vein 15 into the intracardiac opening 2 in a partition wall 22 of the heart 16. For this purpose, guide wires or guide catheters not shown in more detail are used.

Then the shaft 14 is pulled back, wherein the hollow body 3 is pushed out from the holder 12 by the push-and-pull element 11 and widens in the way shown in FIG. 3 and contacts the edge 17 of the opening 2.

Here, the edge 17 causes a deformation of the individual bars 4 directed into the interior of the hollow body 3 in the touching region 18. The illustrated deformation is also produced by internal stress of the individual bars directed outward.

Because the metal cage of the hollow body 3 can be recognized easily in an X-ray or ultrasound image, the size of the opening 2 can be determined from the deformation of the individual bars 4.

For the comparison of orders of magnitudes, markings 19 that are visible on the individual bars 4 in FIG. 1 are formed that can be used as a reference scale. Because the size of the opening 2 must be measured only approximately for determining a fitting closure, the change of the distance of the markings 19 from each other due to the deformation of the individual bars 4 shown in FIG. 3 is insignificant.

Additional markings 20 at a distance to each other in the axial direction are constructed on the push-and-pull element 11 as sections of increased thickness that are also easily visible in the X-ray or ultrasound image and can also be used as a reference scale for determining the size of the opening 2.

Here, two of the markings 20 are arranged so that they bound a holding element 21 formed as a perforated sealing plate in the pulled-back position of the hollow body 3 shown in FIG. 2 and thus secure the hollow body 3 in the holder 12 against unintentional slippage and blockage, for example, in the vein 15.

For the device 1 for measuring the diameter of an intracardiac opening 2, a hollow body 3 made from a metal-wire mesh is provided that has twisted individual bars forming the jacket of the discontinuous wall 5 of the hollow body 3 and forming longitudinal stabilization between two elastically deformable metal-wire meshes 6 forming the end of the hollow body 3 and are deformable in the position of use by the edge 17 of the opening 2 in a touching region into the interior of the hollow body 3. Thus, with the device 1, the size of the opening 2 can be determined, in that, in an X-ray or ultrasound image, the deformation of the individual bars 4 is comparable through the opening 2 with markings 19, 20 arranged on the hollow body 3 and/or on the device 1 adjacent to the hollow body 3.

1. Device (1) for measuring the size of an intracardiac opening (2), comprising a hollow body (3) that has an elastic wall (5) and is insertable into the opening (2) and can expand perpendicular to an insertion direction and having at least two transparent markings (19, 20) arranged at a predetermined distance to each other and the distance can be compared with a molding of the hollow body (3) caused by an edge (17) of the opening (2) to be measured, the hollow body (3) is permeable to liquid and is constructed as a metal cage, the hollow body (3) is discontinuous in a middle region between end sides (7, 8) thereof and the discontinuity is spanned by individual bars (4) that are each formed by at least two twisted wires (23).

2. The device according to claim 1, wherein the individual bars (4) are arranged parallel to each other and to a longitudinal middle axis of the hollow body (3).

3. The device (1) according to claim 1, wherein the hollow body (3) is made from at least one of an elastic material or a memory metal.

4. The device (1) according to claim 1, wherein the hollow body (3) has, in a measurement region, several of the individual bars (4) describe a cylindrical lateral surface and are sufficiently stable for stabilizing the hollow body (3) and are soft and flexible in the contact region (18) with the edge (17) of the opening (2).

5. The device (1) according to claim 1, wherein, in a position of use, the individual bars (4) are adapted to extend through the opening (2) and contact the edge (17) of the opening (2).

6. The device (1) according to claim 1, wherein the hollow body (3) has wire sections (23) that are twisted with each other and are in touching contact, in a position of use, with the edge (17) of the opening (2).

7. The device according to claim 1, wherein the hollow body (3) comprises a metal-wire mesh (6) that can be deformed elastically in a radial direction against a restoring force from a maximum, expanded position on each of its end sides (7, 8) arranged on both sides of the opening (2) in a position of use.

8. The device according to claim 1, wherein ends (7, 8) of the hollow body (3) are connected by a connection element (9) deformable elastically against a tensile stress and that extends centrally through the hollow body (3).

9. The device according to claim 1, wherein an insertion catheter is provided with a sleeve-shaped receptacle (12) for the hollow body (3) in a radially folded-together or compressed form.

10. The device according to claim 9, wherein the hollow body (3) is mounted on a push-and-pull element (11) and can be moved with the push-and-pull element (11) into the receptacle (12) and out from the receptacle (12).

11. The device according to claim 10, wherein the receptacle (12) is constructed on a shaft (14) of the insertion catheter holding the hollow body (3) in a position of use.

12. The device according to claim 1, wherein the markings (19, 20) are arranged adjacent to the hollow body (3) on at least one of a shaft (14) holding the hollow body (3) for of its feed and for its use or on a push-and-pull element (11) connected to the hollow body (3).

13. The device according to claim 1, wherein the markings (19, 20) are arranged on the hollow body (3) on an individual bar (4).

14. The device according to claim 1, wherein the hollow body (3) is mounted so that it is movable on one front end (7, 8) on a push-and-pull element (11).

15. The device according to claim 1, wherein the hollow body (3) is connected detachably to at least one of a shaft (14) or to a push-and-pull element (11).
